(19) United States
(12) Patent Application Publication
(10) Pub. No.: US 2010/0174359 A1
(43) Pub. Date: Jul. 8, 2010

(54) Methods and Systems for Stent-Valve Manufacture and Assembly

(75) Inventors: Jean-Luc Hefti, Yverdon-les-Baine (CH); Stephane Delaloye, Bulach (CH)

Correspondence Address: Brian P. Hopkins;Mintz, Levin, Cohn, Ferris, Glovsky and Popeo, P.C, Chrysler Center, 666 Third Avenue, New York, NY, 10017, US

(73) Assignee: SYMETIS SA, Lausanne (CH)

(21) Appl. No.: 12/608,350

(22) Filed: Oct. 29, 2009

Related U.S. Application Data
(60) Provisional application No. 61/109,310, filed on Oct. 29, 2008.

(51) Int. Cl. A61F 2/82; A61B 17/10; B23P 11/00

(52) U.S. Cl. 623/1.26; 606/139; 29/428; 606/138

(57) ABSTRACT

Embodiments of the current disclosure provide a bioprosthetic heart valve and methods of assembling and usage of the same.

### RELATED APPLICATIONS

This application claims the benefit of priority to U.S. Provisional Application No. 61/109,310, filed Oct. 29, 2008, which is incorporated herein by Reference in its entirety.

### FIELD

Embodiments disclosed herein are generally directed toward bioprosthetic heart valves and methods of assembly and usage related thereto.

### BACKGROUND

Cardiovascular disease or cardiovascular diseases refers to the class of diseases that involve the heart or blood vessels (arteries and veins). This class of diseases thus refers to any disease that affects the cardiovascular system; any may include atherosclerosis (arterial disease), coronary artery disease, valvular heart disease, ischemic heart disease (IHD), or myocardial ischaemia. These diseases are characterized by reduced blood supply to the heart muscle, usually due to coronary artery disease (atherosclerosis of the coronary arteries). Depending on the symptoms and risk, treatment may be with medication, percutaneous coronary intervention (angioplasty) or conventional open-heart surgery.

Best known of the current techniques for the treatment of severe cardiovascular disease is conventional open-heart surgery, which may be used to perform coronary artery bypass grafting, mitral valve replacement, or aortic valve replacement. Coronary artery bypass grafting is a relatively invasive technique where a thoracotomy is performed to expose the patient's heart, and one or more coronary arteries are replaced with synthetic grafts. Valve replacement is a cardiac surgery procedure in which a patient's aortic or mitral valve is replaced with a different valve. Mitral valve replacement therapy is typically performed when the valve becomes too tight (mitral valve stenosis) for blood to flow into the left ventricle, or too loose (mitral valve regurgitation) in which case blood can leak into the left atrium and back up into the lung. Some individuals have a combination of mitral valve stenosis and mitral valve regurgitation or simply one or the other. Aortic valve replacement is a cardiac surgery procedure in which a patient's aortic valve is replaced with a different valve. The aortic valve can also become leaky (aortic insufficiency/regurgitation) or partially blocked (aortic stenosis).

Aortic valve stenosis is a valvular heart disease caused by the incomplete opening of the aortic valve. The blood flow direction within the heart is controlled by the aortic valve. When the valve is in good working condition, blood flow is not impeded between the left ventricle and the aorta. However, narrowing of the aortic valve impedes the blood flow, which is known as the Aortic valve stenosis or AS.

Various types and configurations of prosthetic heart valves, used to replace diseased natural human heart valves, are known in the art. The actual shape and configuration of any particular prosthetic heart valve is dependent to some extent upon the valve being replaced (i.e., mitral valve, tricuspid valve, aortic valve, and pulmonary valve). In general terms, however, the prosthetic heart valve design attempts to replicate the function of the valve being replaced and thus will include valve leaflet-like structures. With this in mind, prosthetic heart valves are generally classified as either forming relatively rigid leaflets and those forming relative flexible leaflets.

As used throughout this specification, prosthetic heart valves having relatively flexible leaflets (or prosthetic heart valve) encompass bioprosthetic heart valves having leaflets made of a biological material as well as synthetic heart valves having leaflets made of a synthetic (e.g., polymeric) material. Regardless, prosthetic heart valves are generally categorized as having a frame or stent, and those which have no stent. The stent in a stented prosthetic heart valve normally includes a substantially circular base (or stent ring), around which an annular suture material is disposed for suturing the prosthesis to heart tissue. Further, a stent forms at least two, typically three, support structures extending from the stent ring. The support structures are commonly referred to as stent posts or commissure posts and include an internal, rigid yet flexible structure extending from the stent ring, covered by a cloth-like material similar to that of the annular suture material. The stent or commissure posts define the juncture between adjacent tissue or synthetic leaflets otherwise secured thereto. Examples of bioprosthetic heart valves are described in U.S. Pat. No. 4,106,129 to Carpentier et al., and U.S. Pat. No. 5,037,434 to Lane, the teachings of which are incorporated herein by reference in their entirety. These disclosures detail a conventional configuration of three leaflets where one leaflet is disposed between each pair of stent or commissure posts

The mitral valve surgical site is relatively easily accessed, with minimal anatomical obstructions above or away from the implant site. Thus, the surgeon is afforded a large, unobstructed area for locating and maneuvering the handle as well as performing necessary procedural steps (e.g., suturing the annulus suture ring to the heart tissue) with minimal or no interference from the handle and/or mechanism. This mitral valve implant site characteristic allows the currently available prosthetic mitral valve holder to assume a relatively bulky and complex form.

Aortic prosthetic heart valve implantation presents certain constraints distinct from those associated with mitral valve replacement. In particular, with aortic heart valve implantation, a surgeon is often faced with little room to maneuver. Depending upon the type of aortotomy performed, the surgeon may first have to pass the prosthesis through a restriction in the aorta known as the sinotubular junction, which is often times smaller than the tissue annulus onto which the prosthetic heart valve will be sutured. The surgeon must then seat the prosthetic heart valve securely in or on the tissue annulus with downward pressure. The surgeon must then tie down all annular sutures (via knots), ensuring that a hemostatic seal is made. Finally, the surgeon must cut-off all sutures in close proximity to the knots. Relative to the orientation of the aortic prosthetic heart valve during the implant procedure, the stent posts extend proximally toward the surgeon (as opposed to the distal stent post direction associated with mitral valve replacement). Thus, while the concern for snagging of the stent posts (i.e., inadvertently looping sutures about stent post(s)) is minimal during aortic prosthetic heart valve implantation, the proximally extending stent posts associated with the stented prosthesis interfere with the various other maneuvers required of the surgeon.

In an effort to more nearly recreate the force distribution along the leaflets of natural tissue valves, some previously known valve designs include circular base portions having longitudinal projections that function as anchors for the commissural points, such as described in U.S. Pat. Nos. 5,844,601, and 6,582,462. While the valve prostheses of earlier patents listed here may readily be collapsed for delivery, those designs are susceptible to problems once deployed. For example, the longitudinal projections of such prostheses may not provide sufficient rigidity to withstand compressive forces applied during normal movements of the heart. Deformation of the commissural anchors may result in varied forces being imposed on the commissures and leaflets, in turn adversely impact functioning of the leaflets. In addition, because the exteriors of the foregoing valve prostheses are substantially cylindrical, the prostheses are less likely to adequately conform to, and become anchored within the valve annulus anatomy during deployment. As a result, cyclic loading of the valve may result in some slippage or migration of the anchor relative to the patient's anatomy.

Currently, high risk or inoperable patient suffering from severe aortic stenosis may be treated by minimally invasive or percutaneous implantation of an aortic bioprosthesis. Usually this kind of bioprosthesis comprises a valve element (e.g. biological porcine valve or porcine/bovine/equine pericardium valve, synthetic valve or others) for regulating the blood flow and a stent (balloon expandable, self expandable or others) for holding the valve element and anchoring it within or at the native aortic annulus.

For handling reasons, the valve may be sutured within the stent having a large circumference Dm (stent circumference during manufacturing). However, the assembled valve/stent has to be crimped to a smaller circumference Dc (stent circumference after crimping) onto a delivery catheter allowing positioning/delivery of the bioprosthesis at the intended implant location. Since the expandable stent should create a certain radial force when implanted, the circumference of the prosthesis after implantation is Di, whereas Dm>Di>Dc.

The difference between the circumferences Dm, Di and Dc may influence the bioprosthesis function of the current disclosure. The valve may be designed to have optimal performances at an implanted circumference Di, which is smaller than the circumference Dm. Such valves function sub-optimally at circumference Dm and optimally at circumference Di. This difference in circumference may be adjusted by adjusting the geometry of the commissural leaflets, which is currently practiced for pericardium based values. However, most biologic valves based on native leaflets may not offer the possibility of adjusting their geometry to compensate for the circumference difference, e.g., by trimming or re-shaping the cusps may reduce the durability of the valves. The embodiments of the current disclosure provides a bioprosthetic heart valve that obviates the requirement for adjusting the geometry of the valve.

In view of the foregoing, it would be desirable to provide a valve that is capable of conforming to a patient's anatomy while providing a uniform degree of rigidity and protection for critical valve components. It therefore would be desirable to provide a valve prosthesis having portions that are capable of deforming circumferentially to adapt to the shape of the pre-existing valve annulus, but which is not susceptible to deformation or migration due to normal movement of the heart. Still further, it would be desirable to provide a valve prosthesis having a multi-level component that is anatomically shaped when deployed, thereby enhancing anchoring of the valve and reducing the risk of migration and perivalvular leaks.

### SUMMARY

Some embodiments of the present disclosure are directed to implantable prosthetic heart valves with flexible leaflets. Particularly, the disclosure relates to a prosthetic heart valve system including a device, for example a catheter, for effectuating prosthetic heart valve stent post deflection during implantation thereof. The current disclosure provides a bioprosthetic heart valve and methods of assembling and usage of the same.

Some embodiments of the present disclosure are directed to a system for assembling a stented, bioprosthetic heart valve, including suturing means for suturing a biological valve coupled with a stent, where adjacent commissural portions of the biological valve are sutured together at a predetermined first distance S1. The biological valve of the disclosure may include an initial circumference D1, the stent may include an initial circumference Dm, and means for removing one or more sutures from the adjacent commissural portions of the biological valve, which may result in adjacent commissural portions of the valve being sutured together a second predetermined distance S2. At S2 the assembled stented, bioprosthetic valve includes a circumference D2, where D2 may be (in some embodiments, preferably) greater than D1, and Dm may be substantially equal to D2; where the leaflets of the biological valve may include a coaptation circumference Di after implantation, and Di may be less than Dm.

The system according to some embodiments of the disclosure provide that circumference D2 may be larger than the leaflet coaptation circumference Di, where preferably, Di provides optimal aortic function to the bioprosthesis. The disclosure further provides that the difference between D2 and Di may be about 2-5 mm

Some embodiments of the present disclosure are directed to a system for assembling a stented, bioprosthetic heart valve, including suturing means for suturing a biological valve coupled with a stent, where adjacent commissural portions of the biological valve are sutured together at a predetermined first distance S1. The biological valve of the disclosure may include an initial circumference D1. The system may also include and means for removing one or more sutures from the adjacent commissural portions of the biological valve.

In some such embodiments, the stent may include an initial circumference Dm. Moreover, in such embodiments, the means for removing one or more sutures from the adjacent commissural portions of the biological valve result in adjacent commissural portions of the valve being sutured together a second predetermined distance S2. At S2, the assembled stented, bioprosthetic valve may include a circumference D2, and D2 may be greater than D1. Dm may be substantially equal to D2, and the leaflets of the biological valve may include a coaptation circumference Di after implantation. In addition, Di may be less than Dm.

Some embodiments of the present disclosure are directed to a system for assembling a stented, bioprosthetic heart valve, including suturing means for suturing a biological valve coupled with a stent, where adjacent commissural portions of the biological valve are sutured together at a predetermined first distance S3. In some such embodiments, the assembled stented, bioprosthetic valve may include a circumference D3, and the stent may include an initial circumference Dm. Dm may sometimes be substantially equal to D3, and the leaflets of the biological valve may include a coaptation circumference Di after implantation. Also, Di may be less than Dm in some embodiments.

The system according to some embodiments of the disclosure provide that circumference D3 may be larger than the leaflet coaptation circumference Di, where Di provides optimal aortic function to the bioprosthesis. The disclosure further provides that the difference between D3 and Di may be about 2-5 mm.

According to some embodiments of the disclosure, a method for assembling a stented, bioprosthetic heart valve is provided, which may include any or more (and preferably several and in some embodiments all) of the following steps. Providing a biological valve having a circumference D1, providing a stent having a circumference Dm, coupling the biological valve to the stent, where adjacent commissural portions of the biological valve may be sutured together at an initial predetermined distance S1. Depending upon the embodiments, in some embodiments, suturing of one or more (and preferably, all) of the adjacent commissural portions enable the coupling of the biological valve to the stent, and in some embodiments, other affixation (either sutures and/or other fixation means familiar to those of skill in the art) may be used to couple the biological valve to the stent (in addition to or in place of the suturing of one or more adjacent commissural portions).

Some method of the embodiments further provide removing one or more sutures from the sutured adjacent commissural portions may result in the adjacent commissural portions being sutured together at a second predetermined distance S2. Some embodiments of the disclosure optionally provides a reinforcing fabric to the valve. An assembled stented, bioprosthetic valve according to some embodiments, includes a circumference D2, where D2 may be greater than D1, Dm may be substantially equal to D2. Moreover, in some embodiments, after implantation of the assembled, stented, bioprosthetic valve, the stented-bioprosthetic valve may include a leaflet coaptation circumference of Di. In some embodiments, Di may be less than Dm. In some embodiments, the reinforcement fabric is PET-fabric.

In some embodiments of the current disclosure, a system for assembling a stented, bioprosthetic heart valve is provided, including suturing means for suturing a biological valve coupled with a stent, where one or more (and preferably all) adjacent commissural portions of the biological valve are sutured together at a predetermined first distance S1. The biological valve may include an initial circumference D1, and the stent may include an initial circumference Dm. In some embodiments, a means for removing one or more sutures from the adjacent commissural portions of the biological valve is provided, which may result in adjacent commissural portions of the valve being sutured together a second predetermined distance S2. Accordingly, the assembled stented, bioprosthetic valve according to some such embodiments may include a circumference D2, where D2 is greater than D1, and Dm may be substantially equal to D2. The leaflets of the biological valve may include a coaptation circumference Di after implantation, where Di may be less than Dm (in some embodiments, preferably). The system may also include means for optionally providing a reinforcing fabric to the valve (which may also be attached via suturing means—e.g., needle and thread).

Some embodiments of the disclosure provide a bioprosthetic heart-valve comprising a biological valve having a plurality of commissural portions, and an expandable stent coupleable with the biological valve. Such a device may include a plurality of suture openings positioned on one or more (and in some embodiments, all) of respective adjacent commissural portions. The plurality of suture openings may extend a first predetermined distance S1. The device may also include a plurality of sutures configured to engage the plurality of suture openings, where the plurality of sutures may extend a second predetermined distance S2 which may be less than S1.

According to some embodiments, the commissural distance of the bioprosthetic heart-valve may be S1, and the circumference of the heart-valve may be D1. In additional embodiments, the commissural distance may be S2, and the circumference of the heart-valve may be D2. According to some embodiments, circumference D2 is greater than D1.

In some embodiments, the bioprosthetic heart-valve according to the disclosure provides that the expandable stent has a circumference Dm, where Dm is substantially equal to D2, and after implantation of the assembled, stented, bioprosthetic valve, valve commissural leaflet coaptation circumference is Di, whereas Dm is larger than Di.

In some embodiments of the present disclosure, the bioprosthetic heart-valve, the circumference D2 is larger than the leaflet coaptation circumference Di by about 2-5 mm, where Di provides optimal aortic function to the bioprosthesis.

In some embodiments of the disclosure, a bioprosthetic heart-valve comprising commissures and an expandable stent is presented, where the valve may be mounted within the stent. When the stent is partially expanded, the valve may have a circumference D1. And when the stent is fully expanded with a circumference Dm, the commissures of the valve may be sutured together along a predetermined distance S2, with the valve having a circumference D2. In some embodiments D2 is substantially equal to Dm and larger than D1.

The bioprosthetic heart valve according to some embodiments of this disclosure, provides that the leaflets of the commissures coapt when the stent is partially expanded and the valve circumference is D1.

According to some embodiments of the disclosure, after implantation the stent is partially expanded and the circumference of the valve is Di, where Di is substantially equal to D1, and at Di the commissural leaflets coapt and provide optimal aortic function to the bioprosthesis.

The bioprosthetic heart valve according to the embodiments of the current disclosure, may have a circumference D2, which may be larger than Di, and Di may be smaller than Dm. The difference between D2 and Di may be about 2-5 mm.

According to some embodiments of the current disclosure, a bioprosthetic heart valve comprising commissures with a plurality of sutures and circumference D2 is presented, where the valve may be assembled within a stent having a circumference Dm, where Dm is substantially equal to D2. The valve assembly within the stent may be obtainable by removing one or more commissural sutures; where before removal of the sutures the circumference of the valve is D1; where D2 is greater than D1. In some embodiments of the disclosure, the valve assembled within the stent may be reinforced with reinforcing fabric.

In some embodiments of the disclosure the reinforcement fabric may be PET-fabric.

In some embodiments of the disclosure, a bioprosthetic heart-valve is presented comprising commissures, where one or more adjacent portions of the commissures are (and in some embodiments, all) sutured together along a predetermined distance S1. In some such embodiments, one or more sutures are removed before (e.g., final) assembling the bioprosthetic valve onto a stent. The circumference of the sutured valve when commissural portions are stitched together may be D1, and the circumference of the sutured valve when one or more commissural sutures are removed may be D2, where the predetermined distance of the extent of the sutures after removal of the one or more sutures may be S2. After implantation, in some such embodiments, the valve has the circumference Di.

The bioprosthetic valve according to some embodiments of the current disclosure may have a circumference D2, which is larger than the implantation circumference Di, and where the difference in circumference preferably provides optimal aortic function to the bioprosthesis.

According to some embodiments of the current disclosure, the bioprosthetic valve may have optimal aortic function, which is achieved by minimizing pressure gradient during systole and absence of leaks during diastole. The valve may provide the best prognosis on minimizing mortality; reduce risk of thromboembolism and hazards of anti-coagulants; reduce need for re-surgery; and provide the most physiological haemodynamic performance.

In further embodiments, the bioprosthetic valve has the circumference before implantation, which may be larger than the circumference after implantation by about 2-5 mm, and may be assembled to a stent.

Some embodiments of the current disclosure describe a bioprosthetic heart-valve comprising a plurality of commissures with commissural portions of the valve sutured together in a predetermined distance S2. The circumference of the sutured valve when one or more commissural portions are stitched together may be D2, and after implantation the valve may have the circumference Di. At Di, the commissural leaflets coapt and provide optimal aortic function to the bioprosthesis.

In some embodiments of the disclosure, a method for assembling a stented, bioprosthetic heart valve is presented, where initially a biological valve may have a circumference D1, and the commissural portions of the valve are initially sutured together at an initial predetermined distance S1. One or more sutures may then be removed, resulting in the commissural portions of the valve being sutured together at a second predetermined distance S2, thus the resulting valve may have a circumference D2, where D2 is preferably greater than D1. This may be followed by reinforcing the valve with a reinforcing fabric; suturing the valve within a stent having a circumference Dm, where Dm is preferably substantially equal to D2, thereby forming a stented, bioprosthetic heart valve.

According to some embodiments of the current disclosure, a method for assembling a stented, bioprosthetic heart valve is provided, where the biological valve may have commissural portions sutured together at a second predetermined distance S2, thereby the resulting valve may have a circumference D2, where D2 may be greater than D1; reinforcing the valve with a reinforcing fabric; suturing the valve within a stent having a circumference Dm, which may be substantially equal to D2, to form the a stented, bioprosthetic heart valve.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to the present invention. Moreover, the description herein of any disadvantages associated with the described products, methods, and/or apparatus, is not intended to limit the invention. Indeed, aspects of the invention may include certain features of the described products, methods, and/or apparatus without suffering from their described disadvantages.

Suturing means may include any device, system, or method, familiar to those of skill in the art to carry out suturing of the biological valve to the stent, and/or suturing of portions of the biological valve together (e.g., adjacent commissural portions). Such means may include (but not limited to) a surgical needle (or surgical-like needle, or ordinary needle for use in sewing tissue together or to something), and surgical thread. Automated and/or motorized sewing equipment may also be used. Moreover, in some embodiments, the suturing means may correspond to an adhesive, and/or the like.

Means for removing sutures from the valve and/or stent, may include any cutting device which can carry out such functionality (e.g., scissors, blade, knife, and the like), and a tool to pull out the sutures (e.g., tweezers), and/or the means for removing sutures may be a combination pliers/cutting/scissors device. Such means may be an automated and/or motorized means, of which, those of skill in the art will understand.

### BRIEF DESCRIPTION OF THE DRAWINGS

In order to understand the invention and to demonstrate how it may be carried out in practice, embodiments will now be described, by way of non-limiting example only, with reference to the accompanying drawings in which:

FIG. 1 shows schematic diagram of a biological valve according to some embodiments with three commissural leaflets 100 supported by commissure base portions 101, extending from base or body. Each cusp 100 terminates at a free edge 102, at the outflow end of valve mechanism. Fixation mechanism in the form of stitches 103 forms a ring or cuff and is designed to be internally seated within a natural valve annulus. The commissural stitches are designed to hold the cusps together with multiple commissural stitches with sutures 103 at a predetermined distance S1, which results in length of the valve with corresponding circumference D1.

FIG. 2 shows biological valve with the commissures cusps 100 stitched together with stitches 103 along a predetermined distance S1, with resultant circumference D1, according to some embodiments.

FIG. 3 shows a schematic diagram of a biological valve with three leaflets or cusps 100 supported by commissure base portions 101, extending from base or body, according to some embodiments. Each cusp 100 terminates at a free edge 102, at the outflow end of valve mechanism. Fixation mechanism in the form of stitches 103 form a ring or cuff and is designed to be internally seated within a natural valve annulus. When one or more of the stitches are removed leaving the sutured cusps at a predetermined distance S2, the resultant length of the valve has the corresponding circumference D2.

FIG. 4 shows a biological valve with the commissures 100 stitched together 103 at a predetermined distance S2, according to some embodiments, where one or more of the stitches were removed from the commissures as in FIG. 1, thus changing the stitching distance from S1 to S2. By removing the stitches the length or distance between the ends of the commissures increases leading to a circumference of the valve corresponding to D2.

FIG. 5 shows biological valve with commissural cusps 100 reinforced with PET-fabric 104 assembled on a stent 105, according to some embodiments.

FIG. 6 shows biological valve with commissural cusps 100, but with no PET-fabric reinforcement, assembled on a stent 106, according to some embodiments. Also shown is the stent support 107.

FIG. 7 shows expandable stents where bioprosthetic heart valve may be mounted 108. The stent has flexible, undulating wire, sometimes called a wireform, 109, according to some embodiments.

### DETAILED DESCRIPTION OF THE PREFERRED EMBODIMENTS

Embodiments of the current disclosure provide heart valve prosthesis, with advantages in terms of durability and haemodynamic properties (for example). In particular, a bioprosthetic stent-valve assembly is disclosed, which retains advantages of common heart valves, and may provide further advantages in terms of durability, haemodynamic properties and ease of surgical insertion.

Some embodiments of the present disclosure are directed to systems, methods, and devices for cardiac valve replacement. These methods, systems, and devices may be applicable to the full range of cardiac-valve therapies including the replacement of failed aortic, mitral, tricuspid, and pulmonary valves. Stent-valves according to some embodiments of the present disclosure may include a valve component and at least one stent component (e.g., a single-stent-valve or a double-stent-valve). The valve component may include a biological or synthetic (e.g., mechanical) valve and/or any other suitable material(s). The stent and valve components may be capable of at least two configurations: a collapsed configuration (e.g., during delivery) and an expanded configuration (e.g., after implantation).

Bioprosthetic Valve

The valve mechanism according to some embodiments of the current disclosure may be a bioprosthetic valve having tissue leaflets or commissural cuffs 100 and may be specifically configured for replacing any heart valve. In general terms, the prosthetic heart valve may include a stent 105, 106, 107, forming stent posts and commissural cuffs 100. The prosthetic valve according to some embodiments of the current disclosure preferably provides for optimum aortic bioprosthesis function with minimized pressure gradient during systole and absence of leaks (paravalvular, central or commissural) during diastole. The optimum aortic bioprosthesis function depends on the dimensions of the implanted bioprosthesis versus dimensions of the bioprosthesis during manufacturing. At optimal valve function, the valve may start to open prior to any detectable forward flow of blood or when there may be a very small pressure gradient across the valve. The initial opening movement of the valve may consist of a rapid phase that may be maximal when the aortic flow is about 75% of the maximum value. The initial opening phase is believed to be assisted by movements of specific parts of the root. Changes in the shape of the root, from the sinotubular junction down to the annulus may occur during the optimal performance of the valve. In some embodiments of the current disclosure, at optimal valve function the valve may consist of a rapid phase that may be maximal when the aortic flow is among other ranges, between 70-80%, or 80-85%, or 85-90%, or more than 90%, and may be any incremental value thereof.

With respect to at least some embodiments of the current disclosure, an optimal bioprosthetic valve and methods for assembling the same are provided.

As used herein the term “bioprosthesis” includes any prosthesis which may be derived in whole or in part from human or other mammals, or organic tissue, which may be implanted into a human. According to embodiments of the disclosure, the term bioprosthesis may include cardiac prostheses such as heart valves, other replacement heart components, and cardiac vascular grafts.

According to some embodiments of the disclosure, a bioprosthetic stent-valve assembly suitable for replacement of the aortic root and also part of the ascending aorta may be made from non-valve material, for example pericardium is provided. The prosthesis may be made in a wide range of sizes. Coronary button holes may be made during surgery to suit the anatomy of the patient. An embodiment of the disclosure provides that the annulus area of the valve is strong, scalloped and made for continuous suturing by extending the outer layer over the leaflets layer. The less bulky annulus enlarges the effective orifice area.

The embodiments of the current disclosure provides bioprosthetic valves, stent-valves, (e.g., which may be used in systems comprising single-stent valves and double-stent valves) and associated methods and systems.

Commissural Leaflets or Cusps

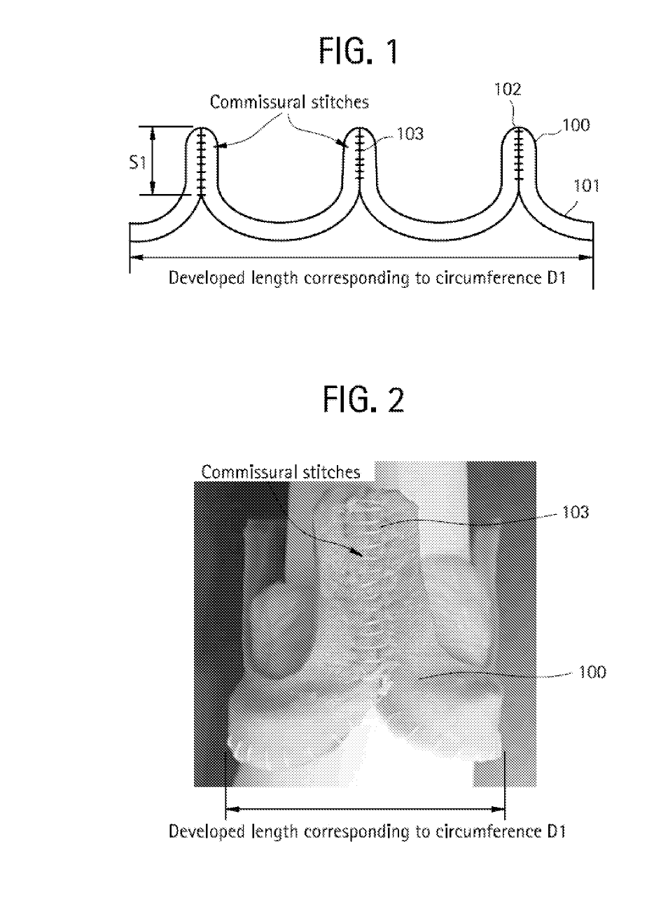

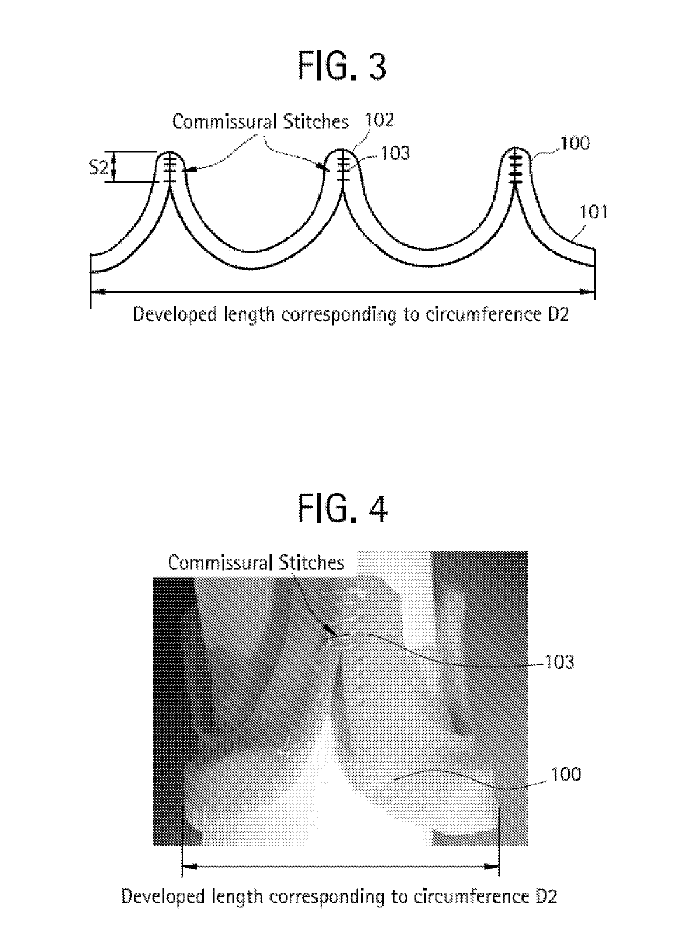

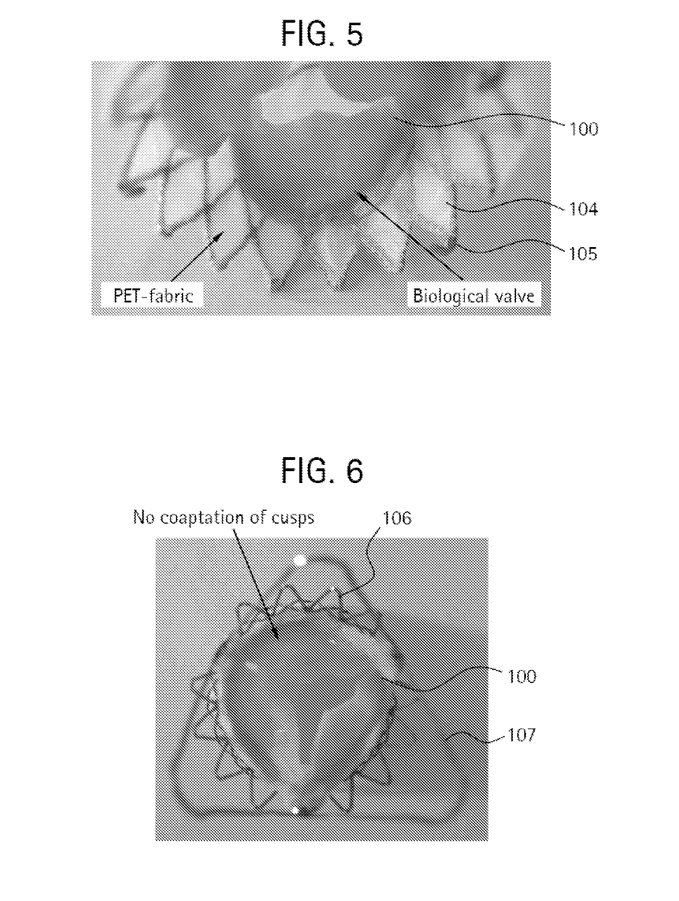

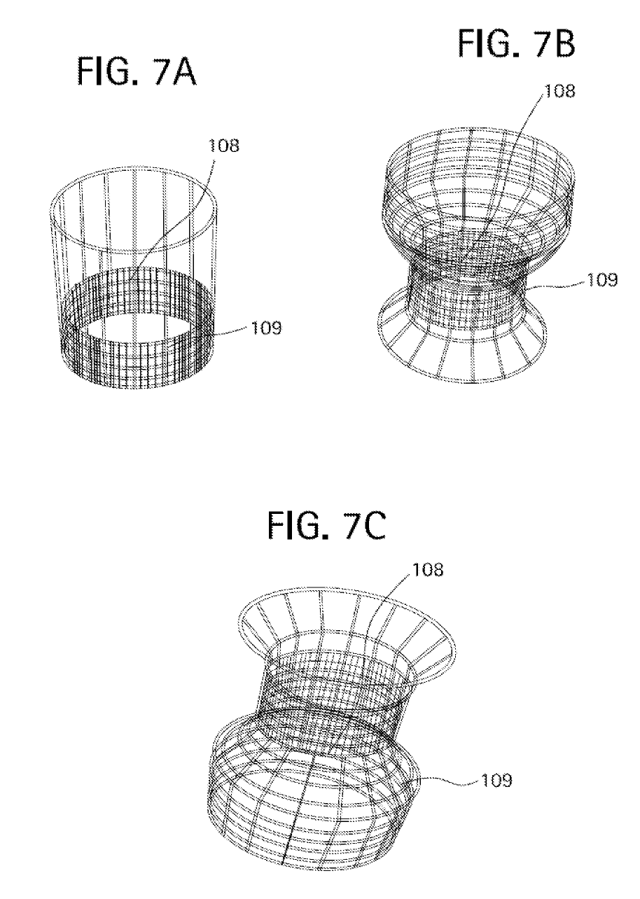

According to some embodiments, the disclosure is directed to implantable prosthetic heart valves with adjustable commissural circumferences. FIG. 1-6 illustrate a prosthetic valve system of the present invention having a valve mechanism with trifoliate configuration having three commissural cusps 100 supported by commissure portions extending from base or body 101 having an outer wall and an inner wall. Each cusp terminates at a free edge 102 at the outflow end of valve mechanism. The commissures of the present disclosure may be made from biological tissues, such as xenograft, autologous, and allograft aortic cusp and pericardium derived materials. In some embodiments, tissue having an elastin content greater than 10% may be utilized, where the tissue may be an anisotopic material. In additional embodiments of the current disclosure, the tissue suitable for use may be from vena cava source material such as porcine, bovine or other large animal vena cava.

In one embodiment, the valve body comprises three leaflets that are fastened together at enlarged lateral end regions to form commissural joints, with the unattached edges forming the coaptation edges of the valve. The leaflets are fastened to a skirt, which is in turn affixed to the stent frame 105, 106, 107. The enlarged lateral end regions of the leaflets permit the material to be folded over to enhance durability of the valve and reduce stress concentration points that could lead to fatigue or tearing of the leaflets.

In some embodiments, the commissural cusps 100 are shaped (and then fixed) into a form that aids their coaptation. In additional embodiments, the outer sheet is also shaped to a desired shape and fixed in the desired shape. In some embodiments of the disclosure, the shaping of the outer sheet (i.e. the change in shape, the stretch or deviation/distortion relative to the initial (unshaped) assembly) is in a portion of the outer sheet on the outflow side of the join between the outer sheet and the cusps or leaflets around the inflow end. Thus, the shaping is preferably in a portion of the outer sheet that corresponds to an aortic sinus in a natural aortic valve. In additional embodiments, the shaping is such that the outer sheet has a conformation resembling that of a natural aortic sinus, for example has the appearance of a bulge when viewed from the exterior of the valve.

In one embodiment of the disclosure, the heart valve prosthesis may have plurality of leaflets encircling a flow opening and of size to coapt to form a valve, each leaflet having a free outflow edge at the outflow end of the leaflet, where the free outflow edge forms a convex (relative to the leaflet) curve in the plane of the leaflet.

According to another embodiment, the disclosure provides a bioprosthetic stent-valve assembly suitable for replacement of the aortic or pulmonary root comprising an outer wall and a plurality of leaflets positioned inside the outer wall, encircling a flow opening and of size to coapt to form a valve, where the outer wall and leaflets maybe formed from natural valve material or material other than natural valve material.

As will be apparent to those skilled in the art, the leaflets of the valve allow flow from the inflow to the outflow end of the valve when in the open position, but in the fully closed position the leaflets coapt to prevent flow back through the valve, i.e. from the outflow end to the inflow end.

According to another embodiment, the disclosure provides that the position of each commissure that provides maximal coaptation is determined by the predetermined distance of the sutures S1 and S2. In removing one or more of the sutures, thereby changing the distance between the commissural cusps, the disclosure provide obtaining heart valves with either circumference D1 or D2. At D1 and after implantation, the commissures may achieve appropriate coaptation. The commissure may include a Commissure Holder that provides maximum coaptation.

The aortic valve cusps, according to embodiments of the disclosure may be biologically inert flaps of tissue, which may function to prevent the flow of blood back into the left ventricle during diastole. In other embodiments, the cusps may be comprised of specific cell types that exhibit specific biological properties.

Commissural Stitches

In an embodiment of the present disclosure, the valve may be assembled by stitching the leaflet sheet to the outer wall (outer sheet and outer protective layer), for example as indicated in FIGS. 1 and 2. The commissures may be formed by stitching through the outer protective layer, outer sheet and the leaflet sheet. The abutted edges of the outer sheet/outer protective layer are sewn together above the level of the top (outflow) edge of the leaflets to complete the encirclement of the flow passage. The inflow edges of the leaflets are sewn onto the outer sheet (or, less preferably, vice versa) and outer protective layer. The stitching may pass through the outer protective layer and the outer sheet. The leaflet sheet and outer sheet are preferably positioned so that the outer sheet extends by a distance between about 1 mm and 4 mm, which may be incremental thereof (e.g., about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 mm,) beyond the inflow edge of the leaflet. The leaflet sheet and outer sheet join at the periphery of the leaflet sheet, so that the leaflet sheet does not extend beyond the joint on the inflow side of the valve.

The joints between the leaflets and the outer sheet may be secured by means of sutures (stitching). According to some embodiment of the disclosure, the commissural stitches may be a monofilament suture, but alternatively can be any other type of suture material, string, rope, wire, polymer strip, or other material known in the art. In some embodiments, the sutures may be durable nylon or polyester thread. Suturing or stitching materials and techniques are described in WO00/59379 and U.S. Pat. No. 5,713,953, which are incorporated herein by Reference in their entireties. Moulding, gluing or welding may be used as an alternative.

According to the embodiments of the current disclosure, the suture line may be configured to maintain its structural integrity when subjected to a tension force, such that the line may effectuate inward deflection of the stent posts. In this regard, the connector assembly may be coupled to the line such that the line defines a loop 106 within the stent that may interconnect the stent posts. A length of the loop may be dictated by an orientation or state of the connector assembly, and can be shortened (or tensioned) to effectuate deflection of the stent posts.

Stitching Distance S1, S2, and S3

At least some of the embodiments of the current disclosure provide commissures that are stitched with sutures 103 at a predetermined distance S1. The predetermined distance S1 may be in the range of.

According to some embodiments of the disclosure, one or more stitches are then removed, which according to some such embodiments, may include the removal of one suture, a pair of sutures 103, more (e.g., 3, 4, 5, 6, 7, 8, 9 or more), but not all sutures, which results in a stitching distance S2. In some embodiments, S2 may be in the range between about 1 mm to 10 mm, and incremental thereof (e.g., 1 mm, about 2 mm, about 3 mm, about 4 mm, about 5 mm, about 6 mm, about 7 mm, about 8 mm, about 9 mm, or about 10 mm, or 1.1, 1.2, and the like). According to some embodiments, the stitching distance is more than 2 mm, more than 3 mm, or more than 4 mm. The predetermined sutures distance S1 may be greater than S2, thus depending on the value of S2, S1 according to embodiments of the current disclosure may be >1 mm, or 1-10 mm, or >10 mm.

According to some embodiments of the disclosure, one or more stitches when removed, according to some such embodiments, may include the removal of one suture, a pair of sutures, more (e.g., 3, 4, 5, 6, 7, 8, 9 or more), but not all sutures, which results in a stitching distance S3. In some embodiments, S3 may be in the range between about 1 mm to 10 mm, and incremental thereof (e.g., 1 mm, about 2 mm, about 3 mm, about 4 mm, about 5 mm, about 6 mm, about 7 mm, about 8 mm, about 9 mm, or about 10 mm, or 1.1, 1.2, and the like). According to some embodiments, the stitching distance is more than 2 mm, more than 3 mm, or more than 4 mm. The predetermined sutures distance S1 may be greater than S3.

Valve Length Circumference D1, D2, and D3

According to some embodiments in the current disclosure, when the predetermined distance of the commissural stitches is S1, the commissures may develop into a valve with circumference of D1. In some embodiments of the disclosure D1 is in the range of 1 mm to 10 mm, and incremental thereof (e.g., 1 mm, about 2 mm, about 3 mm, about 4 mm, about 5 mm, about 6 mm, about 7 mm, about 8 mm, about 9 mm, or about 10 mm, or 1.1, 1.2, and the like). In some embodiments D1 may be less than 1 mm

According to some embodiments of the current disclosure, the removal of one or more of the commissural stitches increases the length of the valve circumference to D2, where D2 is greater than D1 (D2>D1). The measurements of D2, according to the disclosure may be in the range of 1 mm to 10 mm, and incremental thereof (e.g., 1 mm, about 2 mm, about 3 mm, about 4 mm, about 5 mm, about 6 mm, about 7 mm, about 8 mm, about 9 mm, or about 10 mm, or 1.1, 1.2, and the like). In some embodiments D2 may be less than 1 mm In some embodiments, the biological valve functions more optimally when the length is D1 and sub-optimally when the length of circumference is D2.

According to some embodiments of the current disclosure, at the commissural stitches length distance S3, the valve may have the circumference to D3, where D3 is greater than D1 (D3>D1). The measurements of D3, according to the disclosure may be in the range of 1 mm to 10 mm, and incremental thereof (e.g., 1 mm, about 2 mm, about 3 mm, about 4 mm, about 5 mm, about 6 mm, about 7 mm, about 8 mm, about 9 mm, or about 10 mm, or 1.1, 1.2, and the like). In some embodiments D3 may be less than 1 mm. In some embodiments, the biological valve functions more optimally when the length is D1 and sub-optimally when the length of circumference is D3.

Relative Measures of Circumference after Manufacture (Dm) and Circumference after Implantation (Di)

According to embodiments of the current disclosure, the valve within a stent with a circumference Dm may also have a circumference equal or close to Dm (Dm is the stent circumference at manufacturing). For delivery, the assembled valve/stent may be folded into a smaller circumference Dc (stent circumference after crimping) onto a delivery catheter allowing positioning/delivery of the bioprosthesis at the intended implant location. The circumference of the bioprosthesis after the implantation into the heart is Di, where Dm>Di>Dc. In some embodiments of the disclosure Dm is in the range of 1 mm to 10 mm, and incremental thereof (e.g., 1 mm, about 2 mm, about 3 mm, about 4 mm, about 5 mm, about 6 mm, about 7 mm, about 8 mm, about 9 mm, or about 10 mm, or 1.1, 1.2, and the like). In some embodiments Dm may be less the 1 mm Di is in the range of 1 mm to 10 mm, and incremental thereof (e.g., 1 mm, about 2 mm, about 3 mm, about 4 mm, about 5 mm, about 6 mm, about 7 mm, about 8 mm, about 9 mm, or about 10 mm, or 1.1, 1.2, and the like). In some embodiments Di may be less the 1 mm and Dc is in the range of 1 mm to 10 mm, and incremental thereof (e.g., 1 mm, about 2 mm, about 3 mm, about 4 mm, about 5 mm, about 6 mm, about 7 mm, about 8 mm, about 9 mm, or about 10 mm, or 1.1, 1.2, and the like). In some embodiments Dc may be less the 1 mm.

In some embodiments of the disclosure, after removing one or more sutures, the commissural stitch distance may be S2, and the valve may have the circumference D2, which is approximately equal to stent manufactured circumference Dm. At circumference D2 or Dm, the commissural leaflets would lack coaptation due to the larger opening of the commissural stitches, which may render the valve to function sub-optimally. Thus, at Dm or D2, the bioprosthetic stent-valve may have a sub-optimum performance due to the leaflet's lack of coaptation. However, after implantation, the bioprosthesis may have an optimum performance at its intended implantation circumference Di with appropriate leaflet coaptation under aortic back pressure.

In some embodiments of the disclosure, the commissural stitch distance may be S3, and the valve may have the circumference D3, which is approximately equal to stent manufactured circumference Dm. At circumference D3 or Dm, the commissural leaflets would lack coaptation due to the larger opening of the commissural stitches, which may render the valve to function sub-optimally. Thus, at Dm or D3, the bioprosthetic stent-valve may have a sub-optimum performance due to the leaflet's lack of coaptation. However, after implantation, the bioprosthesis may have an optimum performance at its intended implantation circumference Di with appropriate leaflet coaptation under aortic back pressure.

In some embodiments of the current disclosure, a non-assembled valve is stentless, where the commissural portions are initially sutured together in a predetermined stitching distance S2 (according to this embodiment, there is no initial S1 stitching distance, thus no D1). Accordingly, the resulting circumference of the biological (stentless) valve is D2. At this point, the biological valve is sutured into a stent having a circumference Dm, which is substantially equal to D2. In some embodiments, at Dm or D2, the bioprosthesis has a suboptimum performance (see FIG. 6), because of the absence of leaflet's coaptation. However, the bioprosthesis has an optimum performance at its intended implantation circumference Di with appropriate leaflet coaptation under arotic back pressure.

In some embodiments of the current disclosure, a non-assembled valve is stentless, where the commissural portions are initially sutured together in a predetermined stitching distance S3 (according to this embodiment, there is no initial S1 stitching distance, thus no D1). Accordingly, the resulting circumference of the biological (stentless) valve is D3. At this point, the biological valve is sutured into a stent having a circumference Dm, which is substantially equal to D3. In some embodiments, at Dm or D3, the bioprosthesis has a suboptimum performance, because of the absence of leaflet's coaptation. However, the bioprosthesis has an optimum performance at its intended implantation circumference Di with appropriate leaflet coaptation under arotic back pressure.

According to some embodiments of the present disclosure, the circumference difference between Dm and Di may be between 1 mm to 5 mm or 2 mm to 5 mm, or between 3 mm to 5 mm, or between 4 m to 5 mm, between 1 mm to >5 mm. In additional embodiments of the disclosure, the difference may be more than about 5 mm to about 10 mm (e.g., about 6 mm, 7 mm, 8 mm, 9 mm, 10 mm).

According to some embodiments of the present disclosure, the circumference difference between D2 and Di may be between 1 mm to 5 mm or 2 mm to 5 mm, or between 3 mm to 5 mm, or between 4 m to 5 mm, between 1 mm to >5 mm. In additional embodiments of the disclosure, the difference may be more than about 5 mm to about 10 mm (e.g., about 6 mm, 7 mm, 8 mm, 9 mm, 10 mm)

According to some embodiments of the present disclosure, the circumference difference between D3 and Di may be between 1 mm to 5 mm or 2 mm to 5 mm, or between 3 mm to 5 mm, or between 4 m to 5 mm, between 1 mm to >5 mm. In additional embodiments of the disclosure, the difference may be more than about 5 mm to about 10 mm (e.g., about 6 mm, 7 mm, 8 mm, 9 mm, 10 mm).

Reinforcement Material

According to some embodiments of the current disclosure, the bioprosthesis may be covered with a material for reinforcement 105, such material may be any of various suitable biocompatible synthetic materials.

The leaflets and outer wall of the current disclosure may be formed from a sheet material or materials. In some embodiments, the outer wall may be formed from a biological material (other than natural valve material), or a biological material covered or reinforced with a non-biological, biocompatible material. The non-biological material may protect the biological material against dilatation and/or calcification and may also assist in retaining the desired root shape, as discussed further below. The outer protective layer may be formed from a material that is resistant to fixation and preservation solutions such as glutaraldehyde or ethanol. It may be capable of assisting in retaining the root shape determined by the outer sheet and/or of protecting the outer sheet from calcification and/or rupture. The outer protective layer may be porous, so long as the outer sheet is not also porous.

According to some embodiments, the leaflets may be formed from a biological material, for example pericardium. The biological material leaflets may be formed without covering or reinforcement with a non-biological, biocompatible material. Thus, the outer wall may comprise a layer of biological material and a layer of a non-biological, biocompatible material. In some embodiments of the current disclosure, the outer wall may be formed from pericardium covered or reinforced on the outside with a woven fabric, preferably woven polyester (PET), for example Dacron™, or polytetrafluoroethylene (PTFE), or thermoplastic polymer.

The reinforcing material, according to the current disclosure, may be confined to the commissural regions (for example may extend for 5, 4, 3, 2 or 1 mm on either side of a commisure). It is preferred that the width of the reinforcing fabric is 1 to 4 mm, preferably 2 mm. Preferably, it is of less than 3 mm or 2 mm thick in a plane perpendicular to the outer sheet; still more preferably, it is between about 0.5 and about 2 mm thick, and may be incremental thereof (e.g., about 0.6, 0.7, 0.8, 0.9, 1.0, 1.1, 1.2, 1.3, 1.4, 1.5, 1.6, 1.7, 1.8, 1.9, or 2.0 mm, and the like). It is preferred that the reinforcing material does not increase the overall circumference of the valve prosthesis (i.e. the valve prosthesis fits through the same sizing hole in a size gauging device with or without the reinforcing strips). The reinforcement may be woven polyester (PET), for example Dacron™ (DuPont).

In additional embodiments, reinforcement materials may be used that may be amenable to modification, have a controlled degradation, and have properties that may promote cellular population and maintain its integrity. The reinforcement may lack cytotoxicity and not elicit an immune or inflammatory response. According to the disclosure, a suitable reinforcement for a bioprosthetic heart valve may include, but not limited to, collagen, polyglycolic acid, polyhydroxyalkanoate, poly-4-hydroxybutyrate, electrospun poly ureas, and fibrin. These reinforcements may be populated with range of cells, including ovine carotid artery cells, human aortic myofibroblasts, and pericardial fibroblasts. Reinforcement may include, precoating polyglycolic acid with human extracellular matrix proteins to improve the population of the reinforcement and increase attachment of human arotic myofibroblasts.

Methods for Assembling Bioprosthetic Stent-Valve

The disclosure additionally provides embodiments directed to methods for forming a bioprosthetic stent-valve assembly suitable for replacement of the aortic root comprising the steps of forming an outer wall and a plurality of leaflets positioned inside the outer wall, encircling a flow opening and of size to coapt to form a valve, where the outer wall and leaflets are formed from material other than natural valve material.

According to some embodiments of the disclosure, a method for forming a bioprosthetic stent-valve assembly comprises one or more, and preferably several (and in some embodiments, preferably all of) steps of: providing a biological valve having a circumference D1, where commissural portions of the valve are sutured together at an initial predetermined distance S1 in a manner such that the leaflets coapt, then removing one or more commissural sutures resulting in the commissural portions of the valve being sutured together a second predetermined distance S2, and the resulting valve having a circumference D2, where D2 is greater than D1. The valve then is reinforced with a PET-fabric, or other material well known in the art, or disclosed herein, followed by suturing the valve within a stent with a circumference Dm substantially equal to D2 to form the bioprosthetic heart valve. The bioprosthetic heart valve assumes an implantation circumference Di corresponding to the distance D1, which enables an optimum performance with substantially appropriate commissural cusp coaptation under aortic back pressure.

According to some embodiments of the disclosure, a method for forming a bioprosthetic stent-valve assembly comprises one or more, and preferably several (and in some embodiments, preferably all of) steps of: providing a biological valve having a circumference D1, where commissural portions of the valve are sutured together at an initial predetermined distance Si in a manner such that the leaflets coapt, then removing one or more commissural sutures resulting in the commissural portions of the valve being sutured together a second predetermined distance S3, and the resulting valve having a circumference D3, where D3 is greater than D1. The valve then is reinforced with a PET-fabric, or other material well known in the art, or disclosed herein, followed by suturing the valve within a stent with a circumference Dm substantially equal to D3 to form the bioprosthetic heart valve. The bioprosthetic heart valve assumes an implantation circumference Di corresponding to the distance D1, which enables an optimum performance with substantially appropriate commissural cusp coaptation under aortic back pressure.

According to some embodiments of the disclosure, a method for forming a bioprosthetic stent-valve assembly comprises one or more, and preferably several (and in some embodiments, preferably all of) steps of: providing a biological valve by joining commissural portions of the valve together a predetermined distance resulting in a circumference D2 of the valve being larger than a distance leading to coaptation of the leaflets, then reinforcing the valve with a PET-fabric, or other material well known in the art, or disclosed herein, followed by suturing the valve within a stent having a circumference Dm substantially equal to D2 to form the bioprosthetic heart valve. The bioprosthetic heart valve assumes an implantation circumference Di, where Di is less than D2 and where Di includes an optimum performance with substantially appropriate leaflet coaptation under aortic back pressure.

According to some embodiments of the disclosure, a method for forming a bioprosthetic stent-valve assembly comprises one or more, and preferably several (and in some embodiments, preferably all of) steps of: providing a biological valve by joining commissural portions of the valve together a predetermined distance resulting in a circumference D3 of the valve being larger than a distance leading to coaptation of the leaflets, then reinforcing the valve with a PET-fabric, or other material well known in the art, or disclosed herein, followed by suturing the valve within a stent having a circumference Dm substantially equal to D3 to form the bioprosthetic heart valve. The bioprosthetic heart valve assumes an implantation circumference Di, where Di is less than D3 and where Di includes an optimum performance with substantially appropriate leaflet coaptation under aortic back pressure.

According to some embodiments of the disclosure, a method for forming a bioprosthetic stent-valve assembly comprises one or more, and preferably several (and in some embodiments, preferably all of) steps of: assembling the valve, by steps comprising forming a plurality of leaflets joined to encircle a flow passage and of a size to coapt to form a valve, and forming an outer sheet or wall joined to the leaflets around an inflow end and along commissures formed where adjacent leaflets join; after assembly of at least the leaflets and outer sheet or wall of the valve; shaping the leaflets and/or outer sheet or wall to a desired shape; fixing the leaflets and/or outer sheet or wall of the valve in the desired shape.

Some embodiments of the invention provide a method for forming a bioprosthetic stent-valve assembly comprising one or more, and preferably several (and in some embodiments, preferably all of) steps of: forming a plurality of leaflets joined to encircle a flow passage and of a size to coapt to form a valve; forming an outer sheet or wall joined to the leaflets around an inflow end and along commissures formed where adjacent leaflets join. The joint between the outer sheet or wall and the leaflets around the inflow end is at the periphery of the leaflets, and the outer sheet or wall extends by a distance between about 1 and about 4 mm and may be incremental thereof (e.g., about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 mm, and the like) beyond the joint with the leaflets at the inflow end, on the inflow side of the joint, or the joint between the outer sheet or wall and the leaflets around the inflow end is at the periphery of the outer sheet or wall, and the leaflet extends by a distance between about 1 mm and 4 mm and may be incremental thereof (e.g., about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 mm, and the like) beyond the joint with the outer sheet or wall at the inflow end, on the inflow side of the joint.

Stent

U.S. Publication No. 2007-0213813 disclosures stents and stent valves that may be used with the present embodiments. U.S. Publication No. 2007-0213813 is incorporated herein by reference in its entirety. Such stents may be made of any suitable, medical grade material, familiar to those of skill in the art, including titanium, stainless steel, and alloys thereof, as well as temperature sensitive materials (e.g., nitinol, shape-memory alloys)

Bioprosthetic valves may be stented where two or more flexible leaflets are mounted within a metallic or polymeric peripheral support frame 105, 106 that usually includes posts or commissures extending in the outflow direction to mimic natural fibrous commissures in the native annulus. According to some embodiments, a support frame for a stent includes an undulating outflow edge 106 including alternating inflow cusps 104 and outflow commissures 100. The commissures are often flexible and extend generally axially in the outflow direction so as to be fixed at the inflow end and be capable of flexing along their lengths and distributing the forces associated with blood flow. One commonly used peripheral support frame is a flexible, undulating wire, sometimes called a wireform, which has a plurality (typically three) of large radius cusps supporting the cusp region of the flexible leaflets (i.e., either a whole xenograft valve or three separate leaflets). The ends of each pair of adjacent cusps converge somewhat asymptotically to form upstanding commissures that terminate in tips, each extending in the opposite direction as the arcuate cusps and having a relatively smaller radius. This provides an undulating reference shape to which a fixed edge of each leaflet attaches (via components such as fabric and sutures) much like the natural fibrous skeleton in the aortic annulus. One example of the construction of a flexible leaflet valve is seen in U.S. Pat. No. 5,928,281, incorporated by Reference in its entirety. Other support frame constructions exhibit sheet-like tubular shapes but still define undulating commissures and cusps on their outflow ends, such as shown in U.S. Pat. No. 5,984,973 to Gerard, et al., incorporated by Reference in its entirety. Components of the valve are typically assembled with one or more biocompatible fabric (e.g., Dacron) coverings, and a fabric-covered sewing ring is provided on the inflow end of the support frame.

The stent may provide support framework for the prosthetic heart valve and further includes an inner frame member or stent ring encompassed by a cover that otherwise serves as a sewing or suturing annulus or flange. The stent posts extend from the stent ring and each are preferably composed of an internal frame structure encompassed by a cloth covering or other reinforcing material. As is known in the art, the internal structure of each of stent posts may be formed of a stiff but resiliently bendable material. This construction allows the stent posts to be deflected from the orientation with respective free ends deflected inwardly, by external force.

According to some embodiments of the current disclosure, the stent-component of a stent valve may include multiple locking elements for locking the valve onto a catheter, protruding outwardly from an outer surface of the stent component, where each locking element includes a first end adjacent to the outer surface of the stent component and a second end spaced apart from the outer surface of the stent component. The second end of at least a first locking element may be located at a different position along a longitudinal axis of the stent component than the second end of at least a second locking element. For example, in one embodiment, the first locking element and the second locking element may have substantially the same lengths, and the first ends of the first and second locking elements may be positioned at multiple, different levels along the longitudinal axis of the stent component. In another embodiment, the first locking element and the second locking element may have different lengths, and the first ends of the first and second locking elements may be positioned at substantially the same level along the longitudinal axis of the stent component.

Delivery System

U.S. Publication No. 2007-0213813 disclosures stent and stent valve delivery systems that may be used with the present embodiments. U.S. Publication No. 2007-0213813 is incorporated herein by reference in its entirety.

In still other embodiments of the present invention, a stent-valve delivery system is provided. A first assembly is provided that includes an outer sheath and guide wire tubing. The delivery system also includes a second assembly including a stent holder configured for removable attachment to at least one attachment element of a stent-valve. The stent-valve may be positioned over the guide wire tubing of the first assembly. The first assembly and the second assembly may be configured for relative movement with respect to one another in order to transition from a closed position to an open position. In the closed position, the outer sheath may encompass the stent-valve still attached to the stent holder and thus constrain expansion of the stent-valve. In the open position, the outer sheath may not constrain expansion of the stent-valve and thus the stent-valve may detach from the stent holder and expand to a fully expanded configuration.

In some embodiments, the first assembly of the stent-valve delivery system may include a coil-reinforced outer sheath and/or a substantially dome-shaped tip, which may provide resistance to kinking due to the bending moment acting onto the delivery system during positioning within, for example, an aortic arch.

In some embodiments, the stent holder of the delivery system may include proximal and distal components positioned adjacent to one another (i.e., no gap). This may reduce or eliminate the risk of catching or damaging the outer sheath of the first assembly when closing the delivery device.

The stent holder, according to some embodiments of the current disclosure, may include at least one chamfered edge positioned adjacent to at least one attachment pin of the stent holder, where the at least one attachment pin is configured for removable attachment to an attachment element of a stent component. The chamfered edge may assist with the release and expansion of the stent-valve from the stent holder when the stent holder is rotated axially.

In some embodiments, an apparatus is provided for collapsing a circumference of a stent-valve to allow capture of the stent-valve within a sheath of a delivery system. The apparatus may include an elongate, substantially flat strip comprising a slit positioned perpendicular to a longitudinal axis of the strip. The elongate, substantially flat strip may include an end having a height less than a height of the slit, such that insertion of the end into the slit forms a loop. Upon placement of an expanded stent-valve within the loop, pulling the end through the slit causes a reduction of the loop circumference and thereby collapses the circumference of the stent-valve. The elongate, substantially flat strip may be formed from any suitable material including, for example, polymer and metal.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The inventors reserve the right to pursue such inventions in later claims.

What is claimed is:

1. A system for assembling a stented, bioprosthetic heart valve, comprising:
suturing means for suturing a biological valve coupled with a stent, wherein:
adjacent commissural portions of the biological valve are sutured together at a predetermined first distance S1,
the biological valve includes an initial circumference D1,
the stent includes an initial circumference Dm;
and
means for removing one or more sutures from the adjacent commissural portions of the biological valve resulting in adjacent commissural portions of the valve being sutured together a second predetermined distance S2, wherein:
the assembled stented, bioprosthetic valve includes a circumference D2, wherein
D2 is greater than D1, and
Dm is substantially equal to D2; wherein
the leaflets of the biological valve include a coaptation circumference Di after implantation, and
Di is less than Dm.

2. The system according to claim 1, wherein the circumference D2 is larger than the leaflet coaptation circumference Di, wherein Di provides optimal aortic function to the bioprosthesis.

3. The system according to claim 2, wherein the difference between D2 and Di is about 2-5 mm

4. A system for assembling a stented, bioprosthetic heart valve, comprising:
suturing means for suturing a biological valve coupled with a stent, wherein:
adjacent commissural portions of the biological valve are sutured together at a predetermined first distance S3,
the assembled stented, bioprosthetic valve includes a circumference D3,
the stent includes an initial circumference Dm;
Dm is substantially equal to D3,
the leaflets of the biological valve include a coaptation circumference Di after implantation, and
Di is less than Dm.

5. The system according to claim 4, wherein the circumference D3 is larger than the leaflet coaptation circumference Di, wherein Di provides an optimal aortic function to the bioprosthesis.

6. The system according to claim 5, wherein the difference between D3 and Di is about 2-5 mm

7. A method for assembling a stented, bioprosthetic heart valve, comprising:
providing a biological valve having a circumference D1;
providing a stent having a circumference Dm;
coupling the biological valve to the stent, wherein adjacent commissural portions of the biological valve are sutured together at an initial predetermined distance S1;
removing one or more sutures from the sutured adjacent commissural portions resulting in the adjacent commissural portions being sutured together at a second predetermined distance S2; and
optionally providing a reinforcing fabric to the valve;
wherein:
the assembled stented, bioprosthetic valve includes a circumference D2,
D2 is greater than D1,
Dm is substantially equal to D2,
after implantation of the assembled, stented, bioprosthetic valve, include a leaflet coaptation circumference of Di, and
Di is less than Dm.

8. The method according to claim 7, wherein the circumference D2 is larger than the leaflet coaptation circumference Di, wherein Di provides optimal aortic function to the bioprosthesis.

9. The method according to claim 8, wherein the difference between D2 and Di is about 2-5 mm

10. The system according to claim 7, the reinforcement fabric is PET-fabric.

11. A system for assembling a stented, bioprosthetic heart valve, comprising:
suturing means for suturing a biological valve coupled with a stent, wherein:
adjacent commissural portions of the biological valve are sutured together at a predetermined first distance S1,
the biological valve includes an initial circumference D1,
the stent includes an initial circumference Dm;
means for removing one or more sutures from the adjacent commissural portions of the biological valve resulting in adjacent commissural portions of the valve being sutured together a second predetermined distance S2;
and
optionally providing a reinforcing fabric to the valve, wherein:
the assembled stented, bioprosthetic valve includes a circumference D2,
D2 is greater than D1,
Dm is substantially equal to D2,
the leaflets of the biological valve include a coaptation circumference Di after implantation, and
Di is less than Dm.

12. The system according to claim 11, wherein the circumference D2 is larger than the implantation circumference Di, wherein Di provides optimal aortic function to the bioprosthesis.

13. The system according to claim 12, wherein the difference between D2 and Di is about 2-5 mm

14. The system according to claim 11, the reinforcement fabric is PET-fabric.

15. A bioprosthetic heart-valve comprising:
a biological valve having a plurality of commissural portions;
an expandable stent coupleable with the biological valve;
a plurality of suture openings positioned on each respective adjacent commissural portions, the plurality of suture openings extending a first predetermined distance S1; and
a plurality of sutures configured to engage the plurality of suture openings, wherein the plurality of sutures extend a second predetermined distance S2 which is less than S1.

16. The bioprosthetic heart-valve according to claim 15, wherein at the distance S1, the circumference of heart-valve is D1, and at the distance S2, the circumference of the heart-valve is D2, wherein D2 is greater than D1.

17. The bioprosthetic heart-valve according to claim 16, wherein the expandable stent has a circumference Dm, wherein Dm is substantially equal to D2.

18. The bioprosthetic heart-valve according to claim 16, wherein after implantation of the assembled, stented, bioprosthetic valve, valve commissural leaflet coaptation circumference is Di.

19. The bioprosthetic heart-valve according to claim 18, wherein the circumference D2 is larger than the leaflet coaptation circumference Di, wherein Di provides optimal aortic function to the bioprosthesis.

20. The bioprosthetic heart-valve according to claim 19, wherein the difference between D2 and Di is about 2-5 mm.
